(19) United States
(12) Patent Application Publication
(10) Pub. No.: US 2023/0310749 A1
(43) Pub. Date: Oct. 5, 2023

(54) ROCURONIUM BROMIDE PREFILLED SYRINGE

(71) Applicant: Hikma Pharmaceuticals USA Inc., Berkeley Heights, NJ (US)

(72) Inventors: Keith Jarmusik, Bedford, OH (US); Ragheb M. AbuRmaileh, Bedford, OH (US)

(21) Appl. No.: 17/658,068

(22) Filed: Apr. 5, 2022

(51) Int. Cl. A61M 5/31; A61K 31/58; A61K 9/00; A61P 21/02; A61K 47/02; A61K 47/12

(52) U.S. Cl. CPC A61M 5/3134; A61K 31/58; A61K 9/0019; A61P 21/02; A61K 47/02; A61K 47/12

(57) ABSTRACT

Prefilled syringes containing a rocuronium bromide solution, where the prefilled syringes comprise a syringe barrel and a syringe tip comprising cyclic olefin monopolymer (“COP”) or cyclic olefin copolymer (“COC”), and having a plunger rod stopper comprising bromobutyl rubber. The syringes can also comprise a luer adapter integral with the syringe tip, where the syringe barrel, syringe tip and luer adapter are a single element comprising COP or COC. The rocuronium bromide solutions include rocuronium bromide, a tonicity agent, one or more buffering agents, and one or more pH adjusters in an aqueous solution having a pH from about 3.8-4.2. The prefilled syringes containing the rocuronium bromide solutions are storage-stable for extended periods of time at a temperature of 2-8° C., have acceptable physical parameters such as glide force and break force, and make safe and robust connections with needles and needleless luer adapter devices (“NLADs”).

The present invention relates to prefilled syringes containing a premixed rocuronium bromide solution in a syringe comprising a syringe barrel, a syringe tip, a plunger and a plunger stopper, where the barrel and tip comprise a cyclic olefin monopolymer (“COP”) or a cyclic olefin copolymer (“COC”), and the plunger stopper comprises bromobutyl rubber. These prefilled syringes are stable upon long-term storage and can be used in emergency situations in conjunction with a needle or a needleless connector for administration to patients in need.

### BACKGROUND OF THE INVENTION

Rocuronium bromide is a nondepolarizing neuromuscular blocker for use as an adjunct to general anesthesia, to facilitate both rapid sequence and routine tracheal intubation, and to provide skeletal muscle relaxation during surgery or mechanical intubation. It is for intravenous use only.

For years, rocuronium bromide has been available only in 5 ml or 10 ml glass multidose vials containing 50 mg or 100 mg of rocuronium bromide, respectively. As a result, the hospital pharmacy must use standard disposable syringes to withdraw the desired amount of medication from the vial, remove the needle used for withdrawal, attach a syringe cap, label the syringe with the product details, and then transfer the syringes to the operating room suite, emergency room, or ambulance for use. These steps must be repeated regularly according to hospital policy due to the short in-use stability time after withdrawal of the rocuronium bromide solution from the vial.

This process has significant drawbacks. Each of the above-noted steps introduces a risk of microbial contamination. Further, the multiple steps involved in this process introduces the potential for significant medication errors, such as improper doses and improper substitution of a different active pharmaceutical agent. This puts patients at risk of serious injury, or even death. For example, the label for the rocuronium bromide vial product contains a warning that medication errors with respect to that product may result in paralysis, respiratory arrest, and death.

Further, while the rocuronium bromide solution in the glass vial is stable for long-term storage when stored at temperatures from 2-8° C., once the vial has been opened and the syringes filled with the rocuronium bromide solution, the filled syringes have a limited in-use stability time, based on hospital policy.

In an attempt to address these issues, compounding companies, such as 503B outsourcing companies, have manufactured prefilled syringes containing a rocuronium bromide composition, including syringes containing 5 ml or 7.5 ml of a rocuronium bromide composition, where the rocuronium bromide is present at a concentration of 10 mg/ml. These prefilled syringes are made using the vial product as the starting material. The syringes used for these products are typically plastic syringes comprising polyethylene or polypropylene for the syringe barrel and tip, and have a rubber plunger stopper. These prefilled syringes, however, have a limited shelf-life due to stability issues. Typically, the prefilled syringes must be used within at most 90 days of manufacture.

Any syringe component that is in contact with the drug solution has the potential to affect the stability of the solution, and/or be affected by the drug solution. For this reason, many prefilled syringes have glass barrels, as drug solutions in prefilled plastic syringes may adsorb to the syringe walls or chemicals in the plastic may leach into the drug solution over time. However, the FDA has indicated that prefilled glass syringes can become clogged and malfunction during the process of connecting them to needleless IV access systems, and as a result has advised that the use of needleless prefilled glass syringes should be avoided in emergency situations if possible. (See https://www.fda.gov/Drugs/DrugSafety/ucm254215.htm.)

Moreover, because of recent adverse glass syringe connection events, the FDA has recognized that demonstrating conformity to the ISO 11040-4 standard alone does not ensure that glass syringes can be properly connected to connecting devices. Therefore, sponsors who seek to rely on conformity to ISO 11040-4 in regulatory submissions involving glass syringes must submit information from supplemental tests to show that such syringes can be safely connected.

Since the plunger stopper in a prefilled syringe is also in contact with the drug solution stored in the syringe, it too can cause stability issues. For example, in 2015, the FDA warned that certain drug solutions, including rocuronium bromide, stored in Becton Dickinson general use syringes lost potency due to an interaction with the rubber stopper. (See https://www.fda.gov/drugs/drug-safety-and-availability/fda-notifies-health-care-professionals-becton-dickinson-replaced-problematic-rubber-stoppers-its.) In addition, the drug solution itself can cause syringe components like the plunger stopper or tip cap seal to degrade or deform over time, which can negatively affect the syringe integrity and result in contamination or degradation of the drug solution stored in the syringe.

U.S. Pat. No. 10,342,808 (“the '808 patent”) attempts to address these issues. It discloses a rocuronium bromide solution stored in a syringe having a cylinder made from a plastic resin, such as polypropylene or cyclic olefin polymer, in combination with a plunger stopper, or gasket, where the gasket is formed by vulcanized chlorobutyl rubber containing calcined clay, and where the calcined clay is the only inorganic filler. (See the '808 patent at col. 2, lines 30-42.) It further discloses that use of this gasket surprisingly allows for the rocuronium bromide solution in the syringe to remain stable for an extended period of time, without deformation of the gasket. (Id. at col. 2, lines 17-21.)

The '808 patent examples disclose test results for rocuronium bromide solutions in polypropylene syringes with a vulcanized chlorobutyl rubber gasket containing as an inorganic filler either (1) varying amount of calcined clay, (2) calcium carbonate treated with lignin, talc, wet silica, or quartz powder, or (3) a mixture of calcined clay and wet silica, where the syringes were stored at 60° C. and normal humidity for one week. Specifically, Experiment 1 discloses that only the gaskets made of vulcanized chlorobutyl rubber with calcined clay as the sole inorganic filler were undeformed after storage of the filled polypropylene syringes at 60° C. and normal humidity for one week, based on viewing the gaskets through a microscope. Experiment 2 related to stability testing, and discloses that the rocuronium bromide potency in the syringes remained at or above 99.0% for all of the gaskets tested after storage at 60° C. and normal humidity for one week. The '808 patent alleges that “it is possible to preserve the rocuronium bromide injection solution-filled prefilled syringe for a long time.” (Id. at col. 10, lines 37-39.) However, the '808 patent does not provide any test results for solutions stored more than one week.

### SUMMARY OF THE INVENTION

The inventors have discovered that contrary to the disclosure of the '808 patent, it is possible to provide a prefilled syringe containing a rocuronium bromide solution that is stable for extended periods of time without using a vulcanized chlorobutyl rubber stopper (gasket) where only calcined clay is used as the filler. Specifically, the inventors have surprisingly discovered that a prefilled syringe containing a premixed rocuronium bromide solution where the syringe barrel and syringe tip comprise a cyclic olefin monopolymer (“COP”) or a cyclic olefin copolymer (“COC”) and the plunger stopper is made of bromobutyl rubber, has long-term stability. It is also safe for use, including with needles and needleless connectors, such as NLADs. Use of the prefilled syringe of the invention also avoids the issues discussed above involving glass syringes, as well as breakage issues inherent in their use.

### BRIEF DESCRIPTION OF THE DRAWINGS

FIG. 1 shows a prefilled syringe for use in the present invention.

FIG. 2 shows an expanded view of the distal end of a prefilled syringe for use in the present invention.

FIG. 3 shows a prefilled syringe for use in the present invention enclosed within a flexible plastic overwrap.

### DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

The prefilled syringe of the present invention contains a premixed rocuronium bromide solution. Here, “premixed” means that the rocuronium bromide solution is prepared using bulk rocuronium bromide and is filled into the syringe at the time of manufacture, as opposed to being prepared from a FDA-approved sterile solution containing rocuronium bromide, such as those made by 503B outsourcing companies. The premixed rocuronium bromide solution comprises rocuronium bromide, a tonicity agent, one or more pH adjusting agents, one or more buffering agents, and water for injection. The solution may also include one or more preservatives, such as methylparaben, propylparaben, or a combination thereof. In one embodiment, however, the rocuronium bromide solution is free of preservatives.

The rocuronium bromide is present in the prefilled syringe at an initial concentration from about 5 mg/ml to about 15 mg/ml. In one embodiment, it is present at an initial concentration of about 10 mg/ml.

The tonicity agent may be selected from the group consisting of dextrose, mannitol, potassium chloride, and sodium chloride. In some embodiments, the tonicity agent is present in an amount sufficient to make the composition isotonic. In some embodiments, the tonicity agent is present in the composition in an amount sufficient to provide an initial osmolality of from about 240 mOsm/kg to about 340 mOsm/kg, or from about 270 mOsm/kg to about 320 mOsm/kg. Use of the term “initial” with respect to a given parameter such as osmolality, pH, rocuronium bromide content, or impurities refers to that parameter at the time the rocuronium bromide solution is filled into the syringe. In another embodiment, the rocuronium bromide solution in the prefilled syringe has an osmolality of from about 240 mOsm/kg to about 340 mOsm/kg, or from about 270 mOsm/kg to about 320 mOsm/kg, upon storage for up to 12 months, or up to 24 or 36 months, at 2-8° C. In an additional embodiment, the rocuronium bromide solution in the prefilled syringe has an osmolality of from about 240 mOsm/kg to about 340 mOsm/kg, or from about 270 mOsm/kg to about 320 mOsm/kg, upon storage for up to six months at 23-27° C. and 55-65% relative humidity.

In a further embodiment, the tonicity agent is sodium chloride, and is present in an initial amount from about 3 mg/ml to about 4 mg/ml. In yet another embodiment, the sodium chloride is present in an initial amount of about 3.3 mg/ml.

The pH adjusting agent is selected from the group consisting of acidifying agents and alkalizing agents, or combinations thereof. In some embodiments, the pH adjusting agent is selected from the group consisting of one or more of citric acid, acetic acid, hydrochloric acid, sodium hydroxide, sodium citrate, potassium hydroxide, and potassium citrate. In one embodiment, the pH adjusting agent is acetic acid, sodium hydroxide, or a combination thereof. In some embodiments, the pH adjusting agent is present in an amount sufficient to provide an initial pH of about 3.5 to about 4.5, or from about 3.8 to about 4.2. In a further embodiment, the pH of the rocuronium bromide solution in the prefilled syringe is from about 3.5 to about 4.5, or from about 3.8 to about 4.2, upon storage for up to 12 months, or up to 24 or 36 months, at 2-8° C. In one embodiment, the pH of the rocuronium bromide solution in the prefilled syringe is from about 3.5 to about 4.5, or from about 3.8 to about 4.2, upon storage for up to six months at 23-27° C. and 55-65% relative humidity.

The premixed rocuronium bromide solution may also contain one or more buffering agents such as sodium acetate, or sodium citrate. The buffering agent should be present in an amount sufficient to keep the rocuronium bromide solution with the desired pH range. In one embodiment, the buffering agent is sodium acetate, present at an initial concentration of about 1 mg/ml to about 3 mg/ml. In another embodiment, the sodium acetate is present at an initial concentration of about 2 mg/ml.

The premixed rocuronium bromide solution may also contain additional excipients, such as non-aqueous solvents or excipients including cyclodextrins, such as B-cyclodextrins, propylene glycol, polyethylene glycols (“PEGs”) of varying molecular weight, and glycerin. These additional excipients can be present in the composition in amounts up to 80%. Specifically, glycerin can be present in amounts of from about 1.6-70%, by weight of the composition PEG 300 can be present in amounts from about 50-65% by weight, PEG 400 can be present in amounts of from about 11.2-67% by weight, and propylene glycol can be present in amounts from 0.0025-80% by weight.

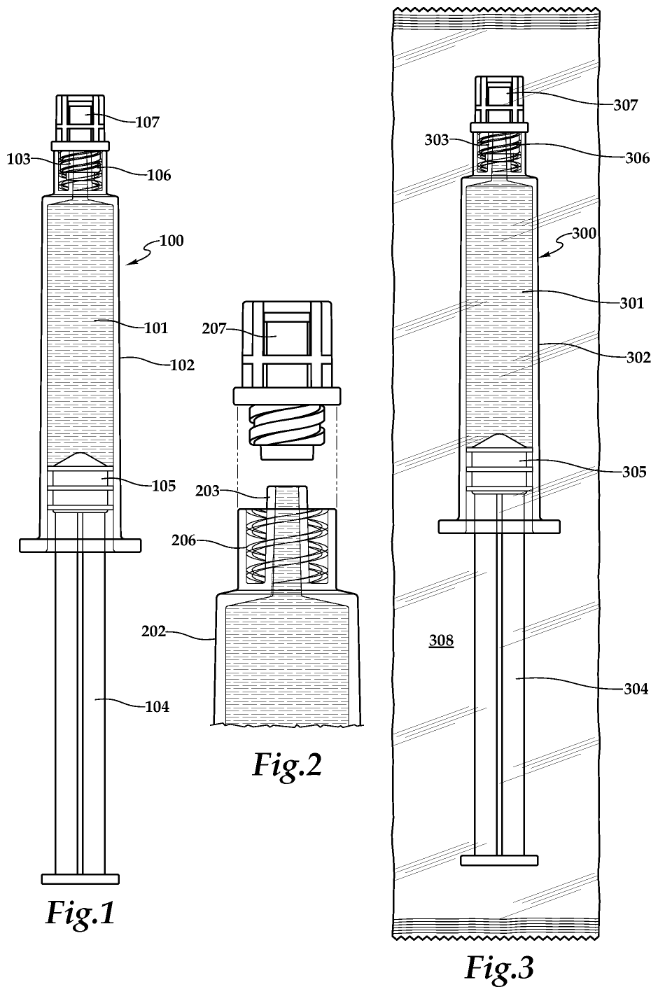

Referring to FIG. 1, the premixed rocuronium bromide solution 101 is contained in a syringe 100 comprising a syringe barrel 102, syringe tip 103, plunger rod 104, and plunger rod stopper 105. The syringe barrel 102 and syringe tip 103 comprise COP or COC, and are preferably a single element. The plunger rod 104 is comprised of a plastic, and in one embodiment comprises polypropylene. In one embodiment, the plunger rod stopper 105 comprises bromobutyl rubber. In another embodiment, the syringe has a luer adapter 106 integral with the syringe tip 103. In a further embodiment, the syringe further comprises a tip cap 107. The tip cap 107 comprises a plastic shell and an elastomer, such as bromobutyl or chlorobutyl rubber, that is fit over the syringe tip 103 by friction or that screws onto the luer adapter 106. In one embodiment, the plastic shell comprises polypropylene. In a further embodiment, the tip cap 107 can comprise an elastomer coated with an poly(ethylene tetrafluoroethylene) (“ETFE”) film. Syringes for use in the present invention include Sterifill Advance™ prefillable syringes, available from Becton-Dickinson, and TopPac® prefillable syringes, available from Schott. Stoppers for use in the present invention include bromobutyl rubber stoppers available from Datwyler, such as the FM457/0 rubber stopper and those available from Aptar, such as the Aptar 6720 rubber stopper. Tip caps for use in the present invention include those available from Aptar, such as the Aptar 6422GS tip cap, where the elastomer comprises chlorobutyl rubber, as well as those available from Datwyler, such as the Datwyler FM257 tip cap, where the elastomer comprises bromobutyl rubber.

In a further embodiment, and as shown for example in FIG. 2, the syringe barrel 202, syringe tip 203, and luer adapter 206 are a single element comprising COP or COC. For example, the syringe barrel, syringe tip, and luer adapter can be made from a single mold. FIG. 2 also shows a tip cap 207 with threads that can be screwed onto the threads on luer adapter 206.

The premixed rocuronium bromide solutions in the prefilled syringes of the invention are stable upon long term storage at 2-8° C. In one embodiment, the solution contains at least about 90%, or at least about 95%, of the initial amount of rocuronium bromide in the solution upon storage for up to 12 months, or up to 24 or 36 months, at 2-8° C., as determined by High Performance Liquid Chromatography (“HPLC”). In a further embodiment, the solution contains at least about 90%, or at least about 95%, of the initial amount of rocuronium bromide in the solution upon storage for up to six months at 23-27° C. and 55-65% relative humidity, as determined by HPLC.

In another embodiment, the total impurities present are not more than 3.5%, or not more than 2%, by weight of the composition, upon storage for up to 12 months, or up to 24 or 36 months, at 2-8° C., as determined by HPLC. In a further embodiment, the total impurities present are not more than 3.5%, or not more than 2%, by weight of the composition, upon storage for up to six months at 23-27° C. and 55-65% relative humidity, as determined by HPLC.

As stated earlier, a concern with the use of plastic for prefilled syringes designed to be storage-stable for an extended period of time is that chemicals in the plastic will over time leach into the composition contained in the syringe. However, the prefilled syringes of the present invention containing a premixed rocuronium bromide solution exhibit only minimal formation of leachables upon extended storage for a period of up to 12 months, or up to 24 or 36 months, at a temperature of 2-8° C. Any such leachables are present in a level below the Permitted Daily Exposure (PDE) with a high Margin of Safety (MOS). As a result, no specifications for leachables are warranted or necessary with respect to the prefilled syringes of the present invention containing a premixed rocuronium bromide solution.

The prefilled syringes of the invention also exhibit superior physical attributes. In one embodiment, upon storage for up to 12 months, or up to 24 or 36 months, at a temperature of 2-8° C., the prefilled syringes have a break force of less than 30 N, or less than 10 N, and a glide force of less than 10 N, when measured according to ISO 11040-6 and ISO 11040-8. Further, and unlike many glass syringes having non-integral luer adapter, the prefilled syringes of the present invention exhibit a safe and robust connection with NLADs. For example, upon storage for up to 12 months, or up to 24 or 36 months, at a temperature of 2-8° C., the prefilled syringes of the present invention having a luer adapter integral with the syringe tip show no evidence of product leakage, air leakage, damage to the syringe, or damages to the NLAD connector when tested according to ISO 80369-7, Small-bore connectors for liquids and gases in healthcare applications—Part 7: Connectors for intravascular or hypodermic applications.

The prefilled syringes containing the rocuronium bromide solution can be provided as a kit, where it is packaged in a clear, flexible plastic overwrap, as shown in FIG. 3. FIG. 3 shows a syringe 300 containing a rocuronium bromide solution 301, the syringe comprising syringe barrel 302, syringe tip 303, plunger rod 304, plunger rod stopper 305, luer adapter 306, and tip cap 307, packaged in a clear flexible overwrap 308. In one embodiment, the overwrap 308 comprises a polyester film, such as that available from Amcor under the tradename Amcor RBA-008. The overwrap protects the prefilled syringes from physical damage resulting from shipping and handling, and also provides evidence of potential product tampering, for example, if the overwrap is open or has a slit or tear in it.

Procedures for filling the rocuronium bromide solution into a syringe, and their subsequent processing, such as aseptic filling conditions, are known in the art. In one embodiment, the rocuronium bromide solution is sterile filtered, and then aseptically filled into the syringes.

The rocuronium bromide solution in the prefilled syringes of the invention can be used for intravenous injection in a bolus dose without dilution. In situations where continuous intravenous infusion is desired, the rocuronium bromide solution in the prefilled syringe may be diluted to a concentration of from about 0.5 mg/ml to about 5 mg/ml in a solution such as 5% Dextrose Injection, USP, 0.9% Sodium Chloride Injection, USP, Lactated Ringers Solution, 5% Glucose In Water, or Sterile Water for Injection. This diluted solution is stable for up to 24 hours when stored at room temperature.

The present invention is further described in the following examples. The examples should not, however, be viewed as limiting the scope of the invention.

### Example 1

A premixed rocuronium bromide solution was prepared in bulk and filled into 5 ml BD Sterifill Advance™ prefillable syringes, with the syringe barrel, syringe tip, and luer adapter being a single element comprising COP, a plunger rod comprising polypropylene, and a Datwyler FM457/0 plunger rod stopper, where the elastomer is bromobutyl rubber. The syringe tip was covered by an Aptar 6422GS tip cap, where the elastomer is chlorobutyl rubber. After the premixed rocuronium bromide solution was prepared, it was sterile filtered through two 0.22 μm PVDF membrane capsule filters. The composition was then aseptically filled into the prefillable syringes. The premixed rocuronium bromide solution is set forth in Table 1 below.

TABLE 1

| Ingredient | Function | Amount |
|---|---|---|
| Rocuronium bromide, USP | Active ingredient | 10 mg/ml |
| Sodium chloride, USP | Tonicity agent | 3.3 mg/ml |
| Sodium acetate trihydrate, USP | Buffering agent | 2 mg/ml |
| Glacial acetic acid, USP | pH adjuster | q.s. to a pH of 3.8-4.2 |
| Sodium hydroxide, NF | pH adjuster | q.s. to a pH of 3.8-4.2 |
| Water for Injection, USP | diluent | q.s. to 1 ml |

### Example 2

Prefilled syringes prepared according to the method of Example 1 were stability tested over a six-month period at 23-27° C. and 55-65% relative humidity. The premixed rocuronium bromide solutions in the syringes were then tested for rocuronium bromide content and for chemical impurities using HPLC. The prefilled syringes containing the premixed rocuronium bromide solutions were also subjected to physical testing, such as break force and glide force, all of which were performed according to ISO 11040-6 and ISO 11040-8. The results are set forth in Table 2 below.

TABLE 2
Accelerated Stability (25° C. +/− 2° C., 60 +/− 5%
RH) for Rocuronium Bromide Injection

| Test | 1M | 3M | 6M |
|---|---|---|---|
| Rocuronium Bromide | 102.9% | 99.5% | 99.3% |
| Osmolality | 293 | 284 | 286 |
| (mOsm/kg) | | | |
| Total Degradants | 0.28% | 0.62% | 1.49% |
| pH | 3.97 | 4.05 | 4.00 |
| Break Force | 6N | 7N | 6N |
| Mean Glide Force | 6N | 6N | 6N |

### Example 3

Prefilled syringes prepared according to the method of Example 1 were stability tested over a 12 month period at a temperature of 2-8° C. The premixed rocuronium bromide solutions in the syringes were then tested for rocuronium bromide content and for chemical impurities using HPLC. The solutions were also tested for sterility using USP <71> and for bacterial endotoxins using USP <85>. The prefilled syringes containing the premixed rocuronium bromide solutions were also subjected to physical testing, such as break force and glide force, both of which were performed according to ISO 11040-6 and ISO 11040-8. The prefilled syringes were also tested for positive pressure liquid leakage, stress-cracking, sub-atmospheric air leakage, resistance to axial load, resistance to unscrewing, and resistance to overriding as set forth in ISO 80369-7, Small-bore connectors for liquids and gases in healthcare applications—Part 7: Connectors for intravascular or hypodermic applications. The results are set forth in Table 3 below.

TABLE 3
Long Term Stability (2-8° C.) for Rocuronium Bromide Injection

| Test | Initial | 1M | 3M | 6M |
|---|---|---|---|---|
| Rocuronium Bromide | 100.9% | 99.7% | 101.1% | 100.4% |
| Osmolality | 286 | 299 | 283 | 285 |
| (mOsm/kg) | | | | |
| Total Degradants | <LOQ | 0.12% | 0.12% | 0.22% |
| pH | 4.05 | 3.96 | 4.08 | 4.03 |
| Break Force | 6N | 6N | 6N | 6N |
| Mean Glide Force | 6N | 6N | 6N | 6N |
| Bacterial Endotoxin | <17.5 | NT | NT | NT |
| | EU/mL | | | |
| Sterility | Sterile | NT | NT | NT |

| Test | Initial | 9M | 12M |
|---|---|---|---|
| Rocuronium Bromide | 100.9% | 101.0% | 99.5% |
| Osmolality | 286 | 279 | 284 |
| (mOsm/kg) | | | |
| Total Degradants | <LOQ | 0.29% | 0.38% |
| pH | 4.05 | 4.07 | 4.00 |
| Break Force | 6N | 6N | 6N |
| Mean Glide Force | 6N | 6N | 6N |
| Bacterial Endotoxin | <17.5 | NT | NT |
| | EU/mL | | |
| Sterility | Sterile | NT | NT |

These results indicate that the rocuronium compositions in the prefilled syringe are chemically stable after storage for a 12 month period at a temperature of 2 to 8° C. Further, the glide force and break force were also acceptable. While not shown in the table, the prefilled syringes showed no evidence of product leakage, air leakage, damage to the syringe, or damages to the NLAD connector after storage for a 12 month period at a temperature of 2° C. to 8° C. when tested according to ISO 80369-7, Small-bore connectors for liquids and gases in healthcare applications Part 7: Connectors for intravascular or hypodermic applications.

These results also indicate that the premixed rocuronium bromide solution in the prefilled syringes of the invention would be chemically stable for a 24-36 month period at a temperature of 2 to 8° C., and that the syringes would also have acceptable glide force, break force, and connectivity parameters upon storage at these same conditions.

### Example 4

The premixed rocuronium bromide solution of Example 1 from the same batch as that of Example 2 was prepared and sterile filtered through two 0.22 μm PVDF membrane capsule filters. The solution was then aseptically filled into standard glass vials. The solutions in the vials were then stability tested for six months at 22-27° C. and 55-65% RH. The rocuronium bromide solutions in the vials were then tested for rocuronium bromide content and for chemical impurities using HPLC. The results are set forth in Table 4.

TABLE 4
Stability Results of Rocuronium Bromide Injection

| Test | 1 M | 3M | 6M |
|---|---|---|---|
| Rocuronium Bromide | 103.8% | 100.4% | 99.5% |
| Osmolality | 303 | 294 | 297 |
| (mOsm/kg) | | | |
| Test | 1M | 3M | 6M |
| Total Degradants | 0.26% | 0.65% | 1.47% |
| pH | 3.93 | 4.01 | 3.98 |

These results indicate the rocuronium bromide solutions in the prefilled syringes of the invention were as stable as those filled into glass vials.

### Example 5

The premixed rocuronium bromide solution of Example 1 from the same batch as that of Example 3 was prepared and sterile filtered through two 0.22 μm PVDF membrane capsule filters. The solution was then aseptically filled into standard glass vials. The solutions in the vials were then stability tested for 12 months at 2-8° C. The rocuronium bromide solutions in the vials were then tested for rocuronium bromide content and for chemical impurities using HPLC. The results are set forth in Table 5.

TABLE 5
Stability Results of Rocuronium Bromide Injection

| Test | Initial | 1M | 3M | 6M |
|---|---|---|---|---|
| Rocuronium Bromide | 101.0% | 101.4% | 100.5% | 101.7% |
| Osmolality | 297 | 311 | 296 | 295 |
| (mOsm/kg) | | | | |
| Total Degradants | <LOQ | 0.18% | 0.10% | 0.21% |
| pH | 4.01 | 3.88 | 4.05 | 3.99 |

| Test | Initial | 9M | 12M |
|---|---|---|---|
| Rocuronium Bromide | 101.0% | 100.7% | 99.5% |
| Osmolality | 297 | 292 | 295 |
| (mOsm/kg) | | | |
| Total Degradants | <LOQ | 0.20% | 0.30% |
| pH | 4.01 | 4.04 | 3.97 |

These results indicate the rocuronium bromide solutions in the prefilled syringes of the invention were as stable as those filled into glass vials when stored at 2-8° C. for 12 months.

### Example 6

In general, analytic protocols for rocuronium bromide and related chemical impurities are well known in the art, and the following HPLC protocol was used with respect to the Examples and are suitable for use herein.

HPLC for Rocuronium Bromide:

Preparation of Buffer: Dissolve about 4.53 g of Tetramethyl ammonium hydroxide pentahydrate in 1000 mL of water.
Adjust the pH to 7.4±0.05 using orthophosphoric acid. Filter the solution through a 0.21 μm filter and degas.
Mobile Phase: Mix Buffer and Acetonitrile in the ratio of 10:90 (% v/v) and degas with aid of sonication for at least 15 minutes.
Standard Solution: Prepare standard stock solution to contain 1 mg/mL of Rocuronium Bromide. Transfer about 50 mg of Rocuronium Bromide USP RS, accurately weighed, into a 50 mL volumetric flask with the aid of 35 mL diluent. Sonicate for about 30 seconds only, complete volume with diluent and mix well. Prepare in duplicate. Standard solutions are stable for 2 days at room temperature.
Sample preparation: Prepare sample solution to contain 1 mg/mL of Rocuronium Bromide.
Transfer 5.0 mL of Rocuronium bromide injection sample into a 50 mL volumetric flask and add about 35 mL diluent. Sonicate for about 30 seconds only, complete volume with diluent and mix well. Sample solutions are stable for 2 days at room temperature.
Conventional HPLC-DAD system is operated using UV detection at 210 nm with 4 nm Band width and enabled reference wavelength 360 nm with 50 nm band width. 250×4.6 mm, 51 μm column or equivalent.

HPLC for Related Compounds:

Preparation of Buffer: Dissolve about 6.25 g of Tetramethyl ammonium hydroxide pentahydrate in 100 mL of water. Adjust the pH to 6.5±0.05 using orthophosphoric acid. Filter the solution through a 0.2 μm filter and degas.
Mobile Phase: Mix 20 mL of Buffer with 200 mL Methanol well to have a clear solution. Then add 800 mL Acetonitrile, mix well, and degas with aid of sonication for at least 15 minutes.
Standard Stock Solution: Prepare standard stock solution to contain 0.04 mg/mL of Rocuronium Bromide. Transfer about 10 mg of Rocuronium Bromide USP RS, accurately weighed, into a 250 mL with the aid of 150 mL diluent. Sonicate for about 30 seconds only, complete volume with diluent and mix well. Prepare in duplicate.
Working Standard Solution (0.5%): Prepare working standard solution to contain 0.01 mg/mL of Rocuronium Bromide. Transfer 12.5 mL of Stock Solution into a 50 mL volumetric flask. Dilute to volume with diluent and mix well. Prepare a working standard check from a separate standard stock solution. Standard solutions are stable for 2 days at 2-8° C.
Sensitivity Solution (0.05%): Prepare sensitivity solution to contain 0.001 mg/mL of Rocuronium Bromide. Transfer 5.0 mL of Working Standard Solution into a 50 mL volumetric flask. Dilute to volume with diluent and mix well.
Prepare sample solution to contain 2 mg/mL of Rocuronium Bromide. Transfer 4.0 mL of Rocuronium bromide injection sample into a 20 mL volumetric flask and add about 10 mL diluent. Sonicate for about 30 seconds only, complete volume with diluent and mix well. Sample solutions are stable for 2 days at 2-8° C.
Conventional HPLC system is operated using UV detection at 210 nm and a C18, 250×4.6 mm, 5 μm column or equivalent.

### Example 7

Prefilled syringes prepared according to the method of Example 1 were stored at a temperature of 60° C. and ambient relative humidity for one week. After that time, the syringe plunger stoppers were removed and checked for any signs of degradation or deformation using a brightfield light microscope at 189× magnification (Olympus BX51). These syringe plunger stoppers showed no signs of degradation or deformation.

### Example 8

Prefilled syringes prepared according to the method of Example 1 were stored at a temperature of 2-8° C. for 12 months. After that time, the syringe plunger stoppers were removed and checked for any signs of degradation or deformation using brightfield light microscope at 189× magnification (Olympus BX51). These syringe plunger stoppers showed no signs of degradation or deformation.

We claim:

1. A prefilled syringe containing a premixed rocuronium bromide solution,
wherein the rocuronium bromide solution comprises (a) an initial amount of about 5 mg/ml to about 15 mg/ml rocuronium bromide; (b) a tonicity agent comprising sodium chloride, (c) a pH adjuster comprising acetic acid, sodium hydroxide, or a combination thereof, (d) a buffering agent comprising sodium acetate, and (e) water for injection;
wherein the rocuronium bromide solution has an initial pH of from about 3.5 to about 4.5;
wherein the syringe has a syringe barrel and a syringe tip comprising COP or COC, a plastic plunger rod, and a plunger rod stopper comprising bromobutyl rubber; and
wherein the rocuronium bromide solution in the prefilled syringe contains at least 90% of the initial amount of rocuronium bromide after storage at 2-8° C. for up to 12 months, as determined by HPLC.

2. The prefilled syringe of claim 1, wherein the rocuronium bromide solution in the prefilled syringe contains at least 95% of the initial amount of rocuronium bromide after storage at 2-8° C. for up to 12 months, as determined by HPLC

3. The prefilled syringe of claim 1, wherein the rocuronium bromide solution contains not more than 3.5% by weight total impurities, after storage at 2-8° C. for up to 12 months, as determined by HPLC.

4. The prefilled syringe of claim 1, wherein the rocuronium bromide solution is present at an initial concentration of about 10 mg/ml.

5. The prefilled syringe of claim 1, wherein the rocuronium bromide solution is present in a volume of about 5 ml to about 10 ml.

6. The prefilled syringe of claim 1, wherein the rocuronium bromide solution has an initial pH of from about 3.8 to about 4.2.

7. The prefilled syringe of claim 1, wherein the syringe further comprises a luer adapter integral with the syringe tip, and wherein the syringe barrel, syringe tip and luer adapter are a single element comprising COP or COC.

8. The prefilled syringe of claim 1, wherein the rocuronium bromide solution has an initial osmolality of from about 240 to about 340 mOsm/kg.

9. The prefilled syringe of claim 8, wherein the rocuronium bromide solution has an initial osmolality of from about 270 to about 320 mOsm/kg.

10. The prefilled syringe of claim 1, wherein the sodium chloride is present in an initial amount of from about 3 mg/ml to about 4 mg/ml.

11. The prefilled syringe of claim 10, where the sodium chloride is present in an initial amount of about 3.3 mg/ml.

12. The prefilled syringe of claim 1, where the syringe has a glide force of less than 30 N and a break force of less than 10 N after storage at 2-8° C. for up to 12 months.

13. The prefilled syringe of claim 1, where the rocuronium bromide solution is free of added preservatives.

14. A prefilled syringe containing a premixed rocuronium bromide solution,
wherein the rocuronium bromide solution comprises (a) an initial amount of about 10 mg/ml rocuronium bromide; (b) a tonicity agent comprising sodium chloride present in an initial amount of about 3.3 mg/ml, (c) a pH adjuster comprising acetic acid, sodium hydroxide, or a combination thereof, (d) a buffering agent comprising sodium acetate present in an initial amount of about 2 mg/ml; and (e) water for injection;
wherein the rocuronium bromide solution has an initial pH of from about 3.8 to about 4.2;
wherein the syringe has a syringe barrel, a syringe tip, and a luer adapter, where the syringe barrel, syringe tip and luer adapter are a single element comprising COP or COC; a plastic plunger rod; and a plunger rod stopper comprising bromobutyl rubber; and
wherein the rocuronium bromide solution in the prefilled syringe contains at least 90% of the initial amount of rocuronium bromide after storage at 2-8° C. for up to 12 months, as determined by HPLC.

15. The prefilled syringe of claim 14, wherein the rocuronium bromide solution in the prefilled syringe contains at least 95% of the initial amount of rocuronium bromide after storage at 2-8° C. for up to 12 months, as determined by HPLC.

16. The prefilled syringe of claim 14, wherein the rocuronium bromide solution in the prefilled syringe contains not more than about 3.5% by weight total impurities, after storage at 2-8° C. for up to 12 months, as determined by HPLC.

17. A kit comprising:
(a) the prefilled syringe of claim 1; and
(b) tamper-evident flexible plastic packaging enveloping the prefilled syringe, wherein the packaging comprises polyester film.
